(19) United States
(12) Patent Application Publication
(10) Pub. No.: US 2021/0007876 A1
(43) Pub. Date: Jan. 14, 2021

(54) NECK COLLAR

(71) Applicants: Sang Kyung KIM, Chungcheongbuk-do (KR); Mi Soon OH, Chungcheongbuk-do (KR)

(72) Inventors: Sang Kyung KIM, Chungcheongbuk-do (KR); Mi Soon OH, Chungcheongbuk-do (KR)

(21) Appl. No.: 16/504,869

(22) Filed: Jul. 8, 2019

(51) Int. Cl. A61F 5/055; A61F 5/058

(52) U.S. Cl. CPC A61F 5/055; A61F 5/05883

(57) ABSTRACT

There is provided a neck collar including: a ring-shaped lower support being open forward from a user and surrounding the user's neck; a vertical support protruding upward from two open ends of the lower support; and a jaw support protruding in two side directions from the upper end of the vertical support and supporting the user's jaw.

### FIELD

The present disclosure relates to a neck collar and, more particularly, to a neck collar that can be simply worn, can minimize a repulsive feeling of the external appearance, and can be deformed to fit the body shape of a user.

### BACKGROUND

This section provides background information related to the present disclosure which is not necessarily prior art.

In general, a ruptured cervical disk, which is also called a neck disk, occurs when the intervertebral disc (disk) of the cervical vertebrae protrudes and thus compresses the spine or nerve roots due to shock or repeated pressure. This disease is treated by wearing a neck collar for several months together with a physical therapy, a traction therapy, a surgical operation, and the like. Most neck collars on the market up to now, which are medical accessories for patients with serious neck disk diseases, is inconvenient to wear and has a large volume because they have to restrict the movement of the cervical vertebrae.

Meanwhile, the interest in lifestyles and work environments that can cause such a neck disk has been recently increased. As can be seen from research on the actual state that twelve of one hundred of over sixties have large possibility of addiction to a smartphone, most adults are not free from mobile devices. Physical diseases that are the most representative severe problems due to long time use of these mobile devices are forward head posture and straight neck syndrome.

Further, people use one or more various display devices in most modern office environments. When people focus on their work, they unconsciously have bad postures such as protruding forward their neck in many cases.

Excessive lowering the head or protruding the neck causes and aggravate forward head posture and straight neck syndrome. Forward head posture and straight neck syndrome cause a neck disk by damaging the muscles and ligaments around the neck bone.

However, as described above, current neck collars are neck collars for curing patients of serious neck disk disease. These neck collars are made of hard materials and large in size to apply predetermined pressure and traction to the cervical vertebrae, so they are inconvenient to wear and cause a lot of inconvenience in daily life.

That is, a neck disk is being increasingly caused by not damage due to specific temporal accidents such as a car accident, but life and work environments and habits. Accordingly, there is a need for a neck collar for preventing neck disk or reduce the possibility in daily life and work environments, forming a habit of having a correct posture, and treating a slight neck disk.

### DOCUMENTS OF RELATED ART

(Patent Document 1) Korean Patent Application Publication No. 10-2011-0066415

### SUMMARY

An aspect of the present disclosure provides a neck collar having the structure that can be simply worn, and can minimize a repulsive feeling of the external appearance.

An aspect of the present disclosure also provides provide a neck collar that can be deformed to fit the body shape of a user.

This section provides a general summary of the disclosure and is not a comprehensive disclosure of its full scope or all of its features.

In order to achieve the objects, a neck collar according to an aspect of the present disclosure includes: a ring-shaped lower support 110 being open forward from a user and surrounding the user's neck; a vertical support 120 protruding upward from two open ends of the lower support 110; and a jaw support 130 protruding in two side directions from the upper end of the vertical support 120 and supporting the user's jaw.

In the neck collar according to an aspect of the present disclosure, the lower support 110 and the jaw support 130 are made of a material that can be curved or bent by a user.

In the neck collar according to an aspect of the present disclosure, the vertical support 120 is composed of a lower section 121 disposed at a lower portion and an upper section 122 disposed at an upper portion, and the lower section 121 and the upper section 120 are combined with each other by sliding, whereby the height of the jaw support 130 is adjusted.

The neck collar according to an aspect of the present disclosure further includes a pivot 140 disposed between the lower support 110 and the vertical support 120 so that the angle between the lower support 110 and the vertical support 120 can be changed.

In the neck collar according to an aspect of the present disclosure, the lower support 110 is curved downward as it goes from the rear to the front.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is a perspective view showing a first embodiment of a neck collar according to the present disclosure;

FIG. 2 is a view showing the state when a user wears the neck collar of FIG. 1;

FIG. 3 is a side view showing a second embodiment of a neck collar according to the present disclosure;

FIG. 4 is a side view showing a third embodiment of a neck collar according to the present disclosure;

FIG. 5 is a view showing the state when a user wears the neck collar of FIG. 4; and

FIG. 6 is a view showing the state when a user wears a fourth embodiment of a neck collar according to the present disclosure.

### DETAILED DESCRIPTION

Exemplary embodiments of the present invention will now be described in detail with reference to the accompanying drawings.

Hereafter, embodiments of a neck collar according to the present disclosure are described in detail with reference to the drawings.

However, it should be understood that the spirit of the present disclosure is not considered as being limited to the embodiments described below and those skilled in the art may easily propose various embodiments included in the same spirit as the present disclosure through changing and modifying, but the changes and modification are included in the spirit of the present disclosure.

Further, the terms to be used hereafter are selected for the convenience of description and should be appropriately construed as meanings coinciding with the spirit of the present disclosure, not being limited to the meanings in dictionaries when finding out the spirit of the present disclosure.

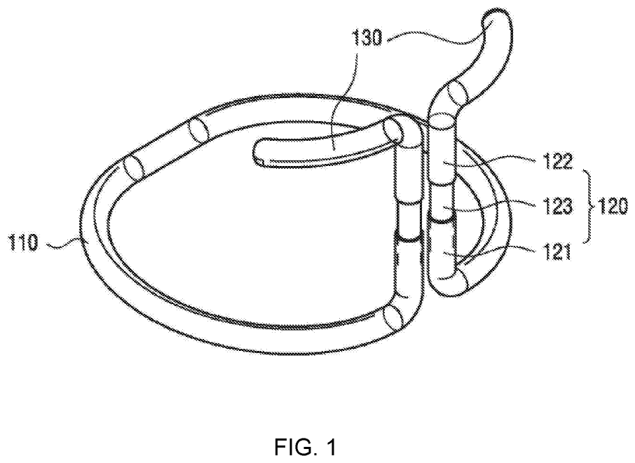

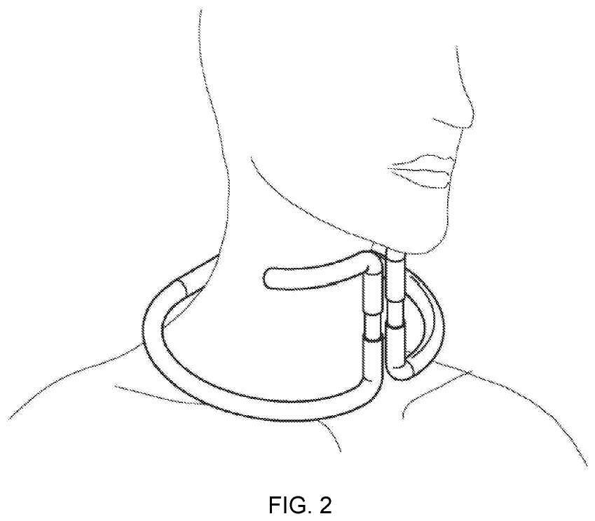

FIG. 1 is a perspective view showing a first embodiment of a neck collar according to the present disclosure and FIG. 2 is a view showing the state when a user wears the neck collar of FIG. 1.

Referring to FIG. 1, a neck collar according to the present embodiment includes a lower support, a vertical support 120, and a jaw support 130.

The lower support 110 is a ring-shaped structure that is open forward from a user and surrounds the user's neck. The vertical support 120 protrudes upward from two open ends of the lower support 110. The jaw support 130 protrudes in two side directions from the upper end of the vertical support 120 and thus supports the jaw of a user.

Referring to FIG. 2, the neck collar according to the present embodiment can be worn with the lower support 110 seated on both shoulders of a user.

The lower support 110, the vertical support 120, and the jaw support 130 are formed in a ring shape or a rod shape, thereby being able to minimize repulsive feeling of the external appearance. The shape and volume are simple, so it is possible to minimize inconvenience when wearing ordinary clothes.

Further, the lower support 110 and the jaw support 130 may be made of a material that can be curved or bent by a user. Users are different in body shape and shape of body parts. Accordingly, users can adjust the lower support 110 and the jaw support 130 to fit their body shapes when wearing the neck collar.

Further, it is possible to open the two open ends of the lower support 110 when putting on the body collar, so it is possible to repeatedly put and take it on and off at any time.

According, as described above, it is possible to prevent unconscious bad postures in daily life or when working in an office space by easily wearing the neck collar. Further, it is possible to minimize inconvenience when a user wears the neck collar, and it is also possible to minimize a repulsive feeling of the external appearance by simplifying the shape and minimizing the size.

The neck collar is useful for correcting a posture particularly in public transportation such as a subway and a bus without specific nests for mobile devices or books by maintaining the angles of the neck and jaw.

Further, referring to FIGS. 1 and 2, the vertical support 120 of the neck collar according to the present embodiment is composed of a lower section 121 disposed at a lower portion and an upper section 122 disposed at an upper portion, in which the lower section 121 and the upper section 122 can be combined with each other by sliding. Accordingly, it is possible to easily adjust the angles of the neck and head and it is possible to adjust the entire length of the vertical support 120 for different neck lengths of users.

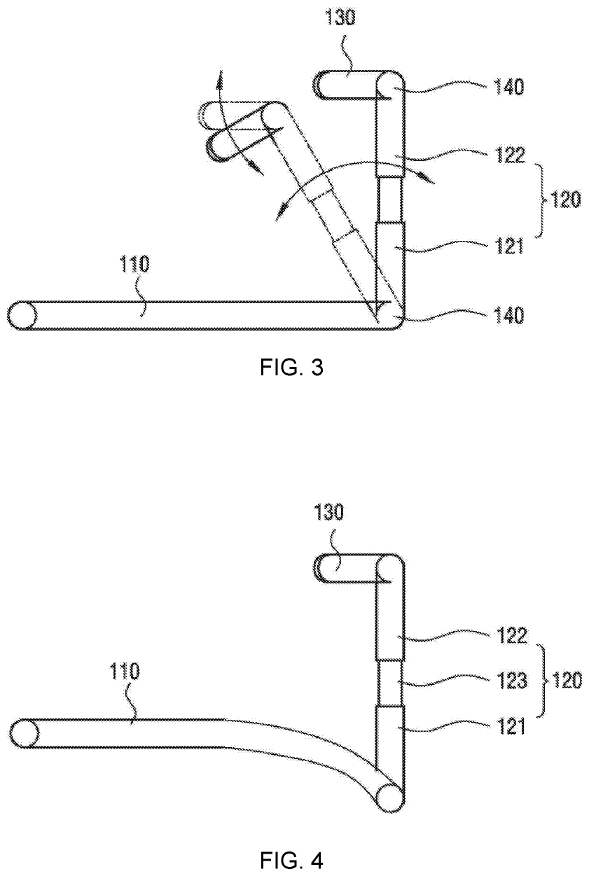

FIG. 3 is a side view showing a second embodiment of a neck collar according to the present disclosure.

Referring to FIG. 3, the neck collar according to the present embodiment may further includes a pivot 140 disposed between the lower support 110 and the vertical support 120. The pivot 140 enables the vertical support 120 to rotate such that the angle between the vertical support and the lower support 110 can be changed. Further, the pivot 140 may be further disposed between the vertical support 120 and the jaw support 130, so the angle between the jaw support and the vertical support 120 can also be adjusted.

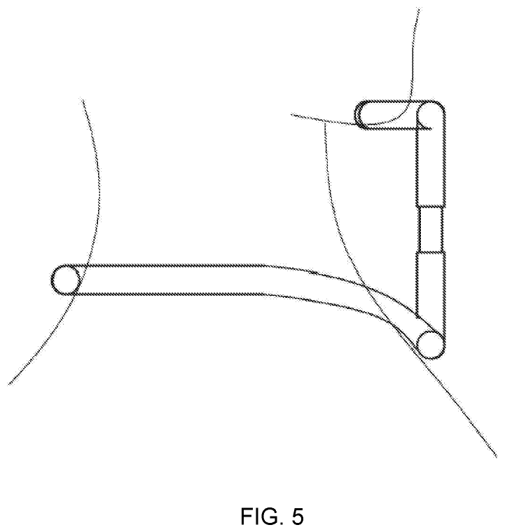

FIG. 4 is a side view showing a third embodiment of a neck collar according to the present disclosure and FIG. 5 is a view showing the state when a user wears the neck collar of FIG. 4.

Referring to FIGS. 4 and 5, it can be seen the lower support 110 of the neck collar according to the present embodiment is curved downward as it goes from the rear to the front.

The jaw support 130 supports downward the user's jaw. When the front of the lower support 110 is pressed downward, the rear of the lower support 110 is stopped on the back of the user's neck, thereby being able to resist the pressure. Further, the front of the curved lower support 110 is held in contact with the upper part of the user's chest, so it is also possible to resist the pressure that is applied from the vertical support 120.

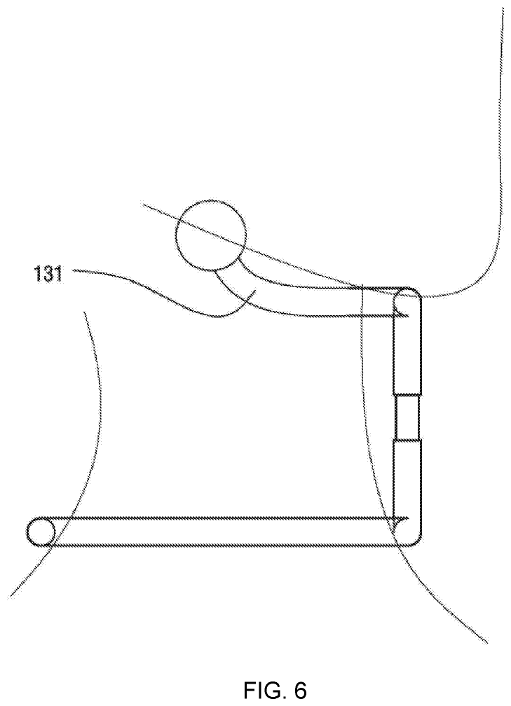

FIG. 6 is a view showing the state when a user wears a fourth embodiment of a neck collar according to the present disclosure.

Referring to FIG. 6, the neck collar according to the present embodiment may further include side guides 131 curved upward to surround the jaw and cheeks of a user with the jaw support extending around both sides of the user's face.

Accordingly, it is possible to prevent an uncomfortable posture by preventing the head of a user from turning when the user takes a rest or a short sleep such as a nap in a chair.

As set forth above, according to exemplary embodiments of the disclosure, it is possible to simply put on a neck collar and minimize a repulsive feeling of the external appearance by employing a linear structure that is brought in close contact with the neck, chest, and jaw.

According to the present disclosure, a material that can be curved or bent is used and a pivot is provided, so a user can deform the neck collar to fit his/her body shape.

What is claimed is:

1. A neck collar comprising:
a ring-shaped lower support being open forward from a user and surrounding the user's neck;
a vertical support protruding upward from two open ends of the lower support;
a jaw support protruding in two side directions from the upper end of the vertical support and supporting the user's jaw; and
a pivot disposed between the lower support and the vertical support so that the angle between the lower support and the vertical support can be changed.

2. The neck collar of claim 1, wherein the lower support and the jaw support are made of a material that can be curved or bent by a user.

3. The neck collar of claim 1, wherein the vertical support is composed of a lower section disposed at a lower portion and an upper section disposed at an upper portion, and
the lower section and the upper section are combined with each other by sliding, whereby the height of the jaw support is adjusted.

4. The neck collar of claim 1, wherein the lower support is curved downward as it goes from the rear to the front.

5. The neck collar of claim 1, wherein the lower support and the jaw support are made of a material that can be curved or bent by a user,
the vertical support is composed of a lower section disposed at a lower portion and an upper section disposed at an upper portion and the lower section and the upper section are combined with each other by sliding, whereby the height of the jaw support is adjusted, and
the neck collar further includes a pivot disposed between the lower support and the vertical support so that the angle between the lower support and the vertical support can be changed, and
the lower support is curved downward as it goes from the rear to the front.
